(19) United States
(12) Patent Application Publication
(10) Pub. No.: US 2006/0110357 A1
(43) Pub. Date: May 25, 2006

(54) Bone putty composition that maintains granule suspension at reduced temperatures

(76) Inventors: Peter A. Materna, Metuchen, NJ (US); Thomas George West, Lawrenceville, NJ (US); Thomas J. Bradbury, Yardley, PA (US)

Correspondence Address: MIDDLETON & REUTLINGER, 2500 BROWN & WILLIAMSON TOWER, LOUISVILLE, KY, 40202, US

(21) Appl. No.: 11/004,878

(22) Filed: Dec. 7, 2004

Related U.S. Application Data
(60) Provisional application No. 60/629,345, filed on Nov. 22, 2004.

(51) Int. Cl. A61K 31/765; A61K 33/42

(52) U.S. Cl. 424/78.38; 424/602

(57) ABSTRACT

The invention is directed to a bone putty composition comprising a carrier formulation comprising water and a carrier substance, and granules wherein said bone putty composition exhibits gel-like Theological properties throughout the temperature range between about body temperature and temperatures approaching about 0° C. The bone putty composition further exhibits reverse phase Theological behavior in at least a portion of the same temperature range. The granules may comprise tricalcium phosphate (hereinafter “TCP”) or other calcium phosphates, they may be porous, and they are suspended in the carrier formulation. In one embodiment of the invention, the bone putty composition withstands exposure to certain temperatures below about room temperature without precipitation of the granules from suspension.

### RELATED APPLICATIONS

This application claims priority to U.S. Provisional Application 60/629,356, filed Nov. 22, 2004 and entitled “Bone Putty Composition That Maintains Granule Suspension At Reduced Temperatures,” which is incorporated herein by reference in its entirety.

### FIELD OF THE INVENTION

This invention is directed to a bone putty composition having rheological properties enabling a suspension of granules to be maintained at temperatures between about room temperature and about body temperature, as well as to certain lower temperatures, without substantial precipitation of the granules from suspension.

### BACKGROUND OF THE INVENTION

Bone putties containing demineralized bone matrix (hereinafter “DBM”) have been made using polyhydroxy compound carrier substances such as glycerol (See, for example, U.S. Pat. Nos. 5,073,373; 5,290,558; 5,484,601). However, one drawback of such formulations is that the putty becomes less viscous and less able to remain positioned inside the body as the putty warms from room temperature to body temperature.

Bone putties containing DBM have also been made using a carrier substance exhibiting reverse phase behavior and having a viscosity which increases with increasing temperature within a limited temperature range. Such a substance is Poloxamer 407, which is a triblock copolymer of ethylene oxide and propylene oxide (also known as Pluronic F127)(BASF, Mt. Olive, N.J.). In such uses, the carrier formulations (e.g., carriers having 20 to 25% by weight Pluronic F127, balance water) provided a non-liquid (such as a gel) at body temperature, and a liquid carrier below room temperature. A tricalcium phosphate (hereinafter “TCP”) putty of that same formulation has also been disclosed, with the TCP being the commercially available product PeriOSS. Use of a carrier formulation which is 40% by weight Pluronic F68 (a similar polymer of lower molecular weight) has also been disclosed.

In commercial-scale manufacturing and distribution, it may not always be possible to control the temperature to which products are exposed during shipping and handling, from the time the product leaves the manufacturer until the time when it is used. Known carrier formulations including reverse phase carriers are such that at certain temperatures corresponding to refrigeration or cold shipping conditions, the formulation attains a low viscosity in which suspended granules of a dense material may not remain suspended, but rather precipitate from suspension. For example, according to data from the manufacturer of Pluronic F127, for concentrations of 22% or lower, at temperatures below 15° C. the viscosity of an aqueous solution is not much greater than the viscosity of water, (i.e., is insufficient to keep dense granules suspended). This is particularly true for suspensions including granules of tricalcium phosphate, which has a greater density than demineralized bone matrix. If precipitation from solution were to occur, end users of the suspension product might not realize that the composition being dispensed is compromised. Depending on the design of the dispenser and its orientation during precipitation, precipitation of a particular granule material from suspension might make it virtually impossible to dispense the precipitate from a syringe.

Accordingly, there is a continuing need for bone putty formulations which provide predictable physical consistency at a variety of temperatures ranging from body temperature to cold temperatures. More particularly, there remains a need for bone putty compositions having gel-like Theological properties which do not thin substantially upon warming from room temperature to body temperature, and which further maintain gel-like rheological properties at temperatures spanning a range between room temperature and cold temperatures. A bone putty formulation of this nature would be particularly useful for preventing precipitation of granules from suspension over a range of temperatures such as might be encountered during shipping.

### BRIEF SUMMARY OF THE INVENTION

The invention is directed to a bone putty composition comprising granules and a carrier formulation which comprises water and a carrier substance, wherein said bone putty composition exhibits gel-like Theological properties throughout the temperature range between about body temperature and temperatures approaching about 0° C. The bone putty composition further exhibits reverse phase Theological behavior in at least a portion of the same temperature range. The granules in the bone putty composition may comprise tricalcium phosphate or other members of the calcium phosphate family. The granules may be porous, and are suspended in the carrier formulation. In one embodiment of the invention, the bone putty composition withstands exposure to certain temperatures down to about 5° C., or even lower, without precipitation of the granules from suspension in the carrier formulation.

### BRIEF DESCRIPTION OF THE FIGURES

FIG. 1 illustrates the boundary between gel and liquid regions as a function of temperature and concentration of Poloxamer 407, and was obtained from BASF Corp., Mt. Olive, N.J.

FIG. 2 illustrates the viscosity of an aqueous solution of Poloxamer 407 as a function of temperature at several concentrations of Poloxamer 407, and was obtained from BASF Corp., Mt. Olive, N.J.

FIG. 3 illustrates one embodiment of packaging for a bone putty composition which provides protection against exposure to a variety of temperatures, for example during shipping.

### DETAILED DESCRIPTION OF PREFERRED EMBODIMENTS

As used herein, “ambient temperature” or “room temperature” shall mean a temperature of about 20° C.

As used herein, “body temperature” shall mean a temperature of about 37.6° C.

As used herein, “reverse phase behavior” shall mean behavior in which viscosity increases with increasing temperature, at least within a certain temperature range.

Viscous solutions have a viscosity, which may be defined at a particular shear rate. Gels have a behavior which exhibits a yield stress. Up to the yield stress, the Theological behavior of a gel is elastic, but beyond that stress the rheological behavior of a gel is viscous or viscoelastic. Both of these behaviors may depend on temperature and the concentration of dissolved substance(s).

As used herein, “porogen” shall mean a substance that is added to a composition at some stage during manufacturing, but is subsequently removed by dissolution or heat treatment, and upon removal leaves void space where the porogen formerly existed.

Heat treatment as used herein shall mean exposure to any temperature above about 100° C.

The bone putty composition of the invention comprises granules suspended in a carrier formulation, said carrier formulation comprising, or alternatively consisting of, an aqueous solution comprising a carrier substance. In one embodiment of the invention, the granules comprise, or alternatively consist of, granules of TCP and the carrier substance comprises, or alternatively consists of, Poloxamer 407.

The bone putty composition of the invention comprises a carrier formulation comprising an aqueous solution of a carrier substance, the carrier formulation exhibiting gel-like consistency throughout the temperature range between about room temperature and about body temperature. The bone putty composition comprises a carrier formulation having a room temperature viscosity or stiffness and a body temperature viscosity or stiffness, wherein the body temperature viscosity or stiffness is at least as large as the room temperature viscosity or stiffness. The carrier formulation further exhibits gel-like consistency down to particular reduced temperatures of between about 5° C. to 10° C., or possibly lower. The particular reduced temperature to which the carrier formulation exhibits gel-like consistency is a function of the concentration and chemistry of the carrier substance used in the carrier formulation.

It is possible that in a certain temperature range, for example below about room temperature, the carrier formulation of the present invention may exhibit reverse phase behavior.

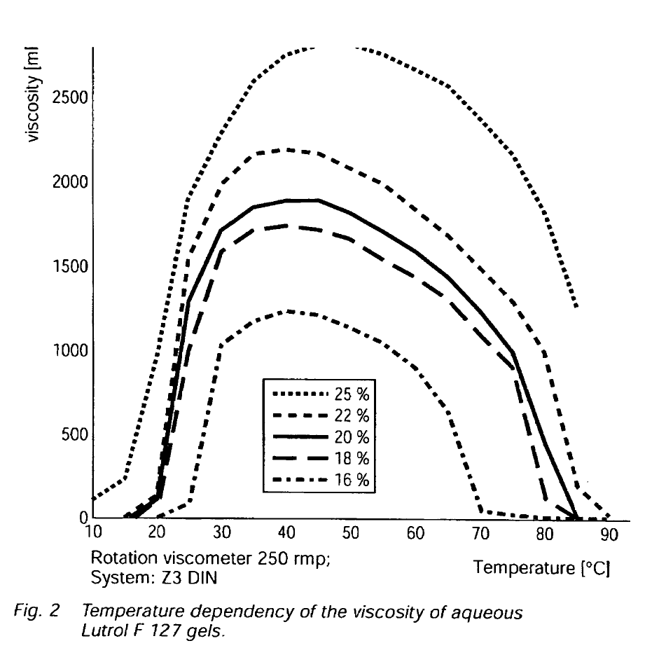

In a preferred embodiment of the invention, the carrier substance comprises, or alternatively consists of, Poloxamer 407 (also known as Pluronic F127) (BASF, Mt. Olive, N.J.). Poloxamer 407 is a polyoxyethylene-polyoxypropylene triblock copolymer having approximately 101 units of ethylene oxide at each end of the molecule and approximately 56 units of a central propylene oxide. The ethylene oxide end blocks are hydrophilic, while the central propylene oxide block is hydrophobic. FIG. 2 illustrates the viscosity profile of an aqueous solution of Poloxamer 407, as a function of the temperature applied to several concentrations of Poloxamer 407.

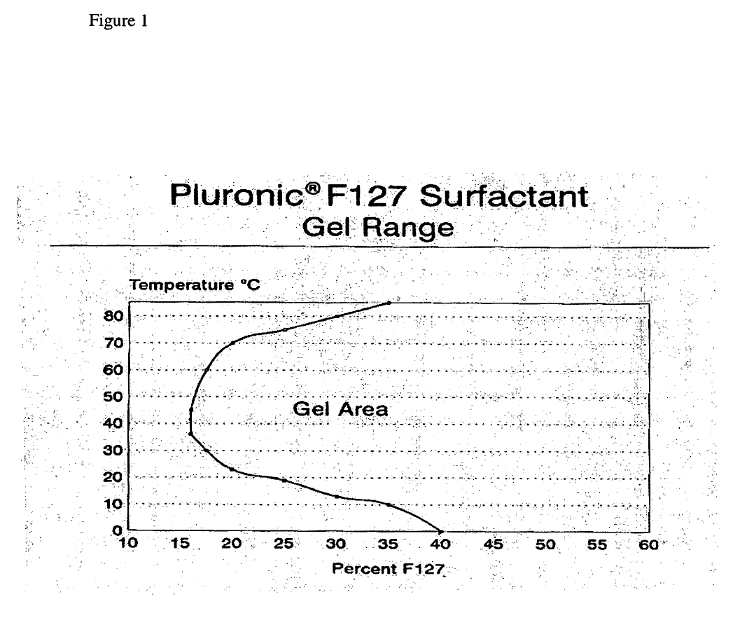

FIG. 1 illustrates the boundary between gel behavior and viscous liquid behavior for Poloxamer 407, as a function of temperature and Poloxamer 407 concentration.

FIG. 1 demonstrates that a concentration of carrier substance Poloxamer 407 of about 35% by weight of the carrier formulation provides a rheological gel behavior at temperatures from about 10° C. to temperatures which are well above body temperature; that a concentration of carrier substance Poloxamer 407 of about 37.5% by weight of the carrier formulation provides a rheological gel behavior at temperatures from about 5° C. to temperatures which are well above body temperature; and that a concentration of carrier substance Poloxamer 407 of about 40% by weight of the carrier formulation provides a Theological gel behavior at temperatures from about 0° C. to temperatures which are well above body temperature. FIG. 1 also indicates that there is another portion of the gel-liquid boundary at temperatures above about 70° C. or about 80° C., but this temperature range is not of interest for the medical applications contemplated for the instant invention.

In an embodiment of the invention, the carrier substance Poloxamer 407 is present in the aqueous carrier formulation at a concentration of about 37.5% by weight of the carrier formulation, with granules as the balance of the bone putty composition. In another embodiment of the invention, the carrier substance Poloxamer 407 may be present in the aqueous carrier formulation at a concentration of about 35% or greater by weight of the carrier formulation, with granules as the balance of the bone putty composition. If it is desired to have a relatively stiff bone putty composition which exhibits the least “runniness” or liquefaction behavior at reduced temperatures below about room temperature or ambient temperature, the carrier substance may be present in the aqueous carrier formulation at a concentration of about 40% by weight of the carrier formulation, with granules as the balance of the bone putty composition.

Bone putty compositions of the invention exhibiting gel-like properties and maintaining those gel-like properties at a range of temperatures between about body temperature and about ambient or room temperature, as well as to certain colder temperatures down to as low as about 0° C., maintain granules contained therein in a suspended state. As discussed elsewhere herein, maintenance of the granules in suspension in the composition at the discussed range of temperatures is an improvement provided by the invention. This improvement provided by the invention and the various embodiments thereof is particularly useful in order to prevent precipitation of the granules from suspension during shipping or transport, which is a time when the bone putty composition may face exposure to temperatures below about ambient or room temperature.

Maintaining suspension of the granules in the composition aids in preventing two undesirable outcomes: (a) the erroneous or unwitting administration of bone putty composition in which the granules, such as for example TCP granules, have precipitated from suspension during transport and are not administered to the patient in need thereof; or (b) the inability to eject material from a syringe due to the precipitation of granules that have preferentially collected near the exit orifice of a syringe. As discussed in greater detail infra, the properties of the carrier substance in the finished product may include a degree of cross-linking and/or an increase in the molecular weight, when compared to the totality of the individual components of the carrier substance prior to manufacture. In one embodiment of the invention, the degree of cross-linking and/or increase in molecular weight may result from irradiation.

The bone putty compositions of the invention comprise granules which include, but are not limited to, TCP or other calcium phosphate compounds. In an embodiment of the invention, the TCP may comprise a large percentage of beta-TCP, such as for example greater than about 50%, or greater than about 75%. This embodiment is desirable due to the in vivo resorption characteristics of beta-TCP.

In another embodiment of the invention, granules useful with the invention are provided in a non-limiting size range of from about 100 micrometers to about 425 micrometers. The granule size range of this embodiment is desirable in order to avoid an immune response in the body, such as for example a leukocyte or macrophage response in the body of a patient.

In another embodiment of the invention, the granules useful with the invention may comprise pores. In a non-limiting hypothesis of the invention, it is believed that porous granules are useful for promoting the ingrowth of bone and related tissues, such as for example vascular tissue. The average size of pores in the granules may range from about 60 microns to about 100 microns, with the sizes of individual pores ranging from about 5 micrometers to about 800 micrometers.

The granules in the bone putty composition of the present invention may comprise about 30.6% by weight of the total weight of the bone putty composition. The final concentration of the granules present in the bone putty composition may be determined based on a determination of the amount of granules to be delivered to the site of administration, as well as a determination of the desired viscosity or rheology of the bone putty composition.

In another embodiment of the invention, therapeutic agents may be added to the bone putty composition in order to further promote the ingrowth of bone tissue and related tissues. Examples of therapeutic agents include, but are not limited to, growth factors such as members of the BMP family, the TGF-beta family, the VEGF family, as well as osteoclastogenesis inhibitors. The therapeutic agents are useful as polynucleotide or polypeptide compositions. Polynucleotide compositions of the invention may take the form of naked plasmid, or as a component of an integrating vector system such as an adenoviral-based gene therapy vector. Polypeptide compositions of the invention may take the form of full length or fragments of the proteins encoded by the gene families described above. In a preferred embodiment of the invention, polypeptide compositions of the invention may be included with the bone putty carrier formulation as mature forms of the full length proteins. Active Pharmaceutical Ingredients may also be included.

The invention further contemplates a kit comprising the bone putty compositions of the invention. The kit may comprise, or alternatively consists of, the bone putty composition of the invention packaged in a manner suitable for delivery or administration via a syringe. The kit may further comprise a syringe suitable for delivery or administration of the bone putty composition to a surgical site. The kit may comprise the bone putty composition packaged in jars or other suitable containers. The kit may comprise the bone putty composition packaged under sterile conditions in one or a plurality of single-use or re-sealable containers. The kit may further comprise the bone putty composition of the invention packaged together with tools or implements suitable for application or administration of the bone putty composition to a surgical site. The kit may further comprise tools or devices for the harvesting, isolating or concentrating of bodily fluids or substances such as for example blood, bone marrow and platelet rich plasma, and for admixture of those substances with the bone putty composition. The kit may also comprise instructions for the preparation of the bone putty composition of the invention prior to administration to the patient(s), or the application or administration of the bone putty composition of the invention to one or more surgical sites of interest.

The granules included in the bone putty composition of the invention may be formed using techniques or processes known in the art, which may include granulation techniques. In a non-limiting exemplary granulation technique, powder(s) may be processed in a fluidized bed granulator in which the powder is fluidized by upwardly moving air, while binder liquid, such as an aqueous solution of polyacrylic acid, may be sprayed into the fluidized powder, thereby causing agglomeration of individual powder particles to each other or to other agglomerates. The longer the process is carried out, the larger the agglomerates become, on average. The process may be carried out for a suitable length of time to produce desirably sized agglomerates.

The powder particles which are supplied to the granulator may comprise precursors for a desired final ceramic substance. For example, the powder may contain hydroxyapatite and calcium pyrophosphate (which may be obtained from Cosmocel, Monterrey, Nuevo León, México) so as to produce tricalcium phosphate (TCP) upon reaction at elevated temperature. Alternatively, or in addition thereto, the powder particles supplied to the granulator may contain particles of a desired final substance.

In addition, the powder supplied to the granulator may further include one or more porogens that may create desired porosity in the eventual granules. Non-limiting examples of porogens useful with this invention include lactose and, in general, almost any solid, decomposable, particulate organic substance. Lactose is a porogen which decomposes into gaseous decomposition products when heated to an appropriate temperature, resulting in voids in a composition. Lactose decomposes at about 220° C. TCP precursor particles may have an average size of about 5 micrometers. Lactose porogen particles may be of a particle size of about 60 micrometers. In one embodiment of the invention, the combined powder used for the fluidized bed granulation process may comprise about 40% by weight lactose and about 60% by weight precursors of tricalcium phosphate.

Following granulation, the agglomerates may be subjected to sintering to form finished granules. Prior to reaching sintering temperatures, the porogen (such as, for example, lactose) and the binder substance (such as, for example, polyacrylic acid) decompose into gaseous decomposition products resulting in void spaces. At temperatures above those necessary to decompose the porogen and binder substances, a reaction of the precursors occurs to form a desired final ceramic product, for example TCP, provided the agglomerates contain combinations of precursor substances suitable to react with each other.

At appropriate temperatures, individual powder particles may sinter to each other. Sintering processes may be performed at peak temperatures of about 1200° C. for about 2 hours. Sintering may result in granules which contain a number of the original powder particles joined to each other, while also containing pores some of which may result from decomposition of the porogen.

The resulting sintered granules may then be size-classified using techniques known in the art, such as for example sieving, to select sintered granules having a size range of from about 100 micrometers to about 425 micrometers. Sieving techniques are known in the art and sieves are commercially available from VWR Scientific (West Chester, Pa.), manufactured to A.S.T.M. Specification E-1. Size classification involving moving air techniques is also possible. The granules may be produced by the just-described process or they may be produced by any other suitable means. The granules used in the compositions and methods of the invention may optionally be porous.

After the granules have been manufactured and (if desired) size-classified, the carrier formulation may be prepared. Preparation of the carrier formulation includes dissolution of the carrier substance(s), such as Poloxamer 407, in water. For dissolution of relatively small concentrations of carrier substance in water, it may be sufficient to use any of a variety of mixing techniques known in the art at any of a variety of temperatures. However, for dissolution of carrier substances present in large concentrations or large quantities, dissolution can advantageously be performed using a mixer, such as for example, a double planetary mixer manufactured by Charles Ross and Son Company (Hauppauge, N.Y.)), preferably at temperatures of about 1° C. to about 4° C. These temperatures are beneficial for dissolution of the carrier substance because carrier substances such as Poloxamer 407 dissolve more readily and have a reduced viscosity at these temperatures, compared to the dissolution and viscosity profile of the same substance at room temperature. Stirring may be performed for appropriate periods of time, which may depend on the concentration of the carrier substance, the size of the batch, and other factors.

In an embodiment of the invention, Poloxamer 407 is the carrier substance and is used to produce an aqueous carrier formulation having between about 35% to about 40% by weight of the aqueous carrier formulation. In order to generate this aqueous carrier formulation, Poloxamer 407 may be sequentially added in increments, with each increment followed by a period of mixing. For example, an initial increment amount of Poloxamer 407 may be added to water to raise the Poloxamer 407 concentration to about 20% by weight of the carrier formulation, followed by dissolution of the carrier substance via mixing. The initial incremental addition is followed by subsequent addition of another incremental amount of Poloxamer 407 sufficient to raise the Poloxamer 407 concentration to about 30% by weight of the carrier formulation, followed by dissolution of the carrier substance via mixing, followed by another incremental addition of the remaining amount of Poloxamer 407 needed to achieve a Poloxamer 407 concentration of between about 35-40% by weight of the carrier formulation, followed by dissolution of the carrier substance via mixing. Dissolution and mixing may be performed at cold temperatures and may be performed sub-atmospheric pressure to reduce the presence of gas bubbles in the putty. It is alternatively possible that the carrier substance could be continuously added during mixing, or added via a combination of batch addition and continuous addition.

Following completion of the dissolution process to form the carrier, the granules may be mixed into the carrier formulation to produce the bone putty composition. Mixing of the granules, such as granules of TCP, into the aqueous carrier formulation may similarly be performed with the use of a double planetary mixer (such as described supra) at a similar range of temperatures. Sufficient mixing time is provided to accomplish thorough and uniform mixing, which may be determined for each individual mixing situation. Mixing may be performed at cold temperatures and may be performed at sub-atmospheric pressure to reduce the presence of gas bubbles in the putty.

It is noted that even at a substantially cold mixing temperature of between about 1° C. to about 4° C., the bone putty composition is sufficiently viscous to allow transfer of the bone putty composition for subsequent processing steps without the granules precipitating from suspension or forming stratified layers. For example, the bone putty composition may be loaded from the pot of the mixer into an intermediate dispensing machine suitable for precise filling of syringes or similar implements.

Following mixing and optional loading into an intermediate dispensing machine, the bone putty composition may be dispensed or loaded into syringes, jars, or any other desired device or container for storage, shipping and/or delivery of the bone putty composition. Filled syringes or containers may then be packaged inside a sterile barrier such as a peel pouch. The bone putty composition and the packaging may then be sterilized using techniques known in the art, such as for example by using a terminal sterilization process. In one embodiment of the invention, the terminal sterilization process comprises, or alternatively consists of, irradiation by gamma irradiation or electron beam irradiation.

In an embodiment of the invention, Gamma irradiation of about 19 kiloGray (minimum) may be used to sterilize the bone putty composition. In another embodiment of the invention, electron beam irradiation parameters of about 26 kiloGray at about 4.5 MeV may be used to sterilize the bone putty composition. The radiation dosage used for the sterilization process may depend, in part, on the amount of microorganisms present during the manufacturing process.

Based on the type of sterilization technique used and the concentration of the carrier substance present in the bone putty composition, the physical attributes of the carrier substance contained in the bone putty composition may be altered following application of the sterilization technique(s).

In one embodiment of the invention, the use of irradiation sterilization techniques on bone putty compositions of the present invention increases the molecular weight of the Poloxamer 407 carrier substance contained in the bone putty compositions relative to non-irradiated control compositions. In a non-limiting hypothesis of the invention, it is believed that some degree of cross-linking of the Poloxamer 407 carrier substance occurs as a result of the irradiation technique(s), thereby increasing the molecular weight of the Poloxamer 407 carrier substance contained in the bone putty composition.

It is further believed that Poloxamer 407 cross-linking provides the beneficial side effect of increasing the viscosity of the bone putty composition at cold temperatures to which the bone putty composition might be exposed post-sterilization such as during shipping, which further aids in the maintenance of suspension of the granules. The effect of irradiation may be detectable as a slight increase in the force needed to expel the bone putty composition from a syringe at room temperature, compared to the force needed to expel non-irradiated bone putty composition.

In a non-limiting embodiment of the invention, the use of Gamma irradiation as described elsewhere herein increases the molecular weight of the carrier substance Poloxamer 407 to between about 16,000 to about 19,000 Daltons, as compared to non-irradiated Poloxamer 407 which has a molecular weight of about 11,000 Daltons.

In another non-limiting embodiment of the invention, the use of electron beam irradiation as described elsewhere herein increases the molecular weight of the carrier substance Poloxamer 407 to between about 13,000 to about 14,000 Daltons, as compared to non-irradiated Poloxamer 407 which has a molecular weight of about 11,000 Daltons.

The production methods of the invention further contemplate the use of Good Manufacturing Practices (“GMP”) procedures to avoid contamination of the final product. One of skill in the art of industrial manufacturing of biologicals and other compositions administered to patients is familiar with the use of GMP procedures in manufacturing.

The shipping of the bone putty composition from manufacturing to customer may include the use of packaging offering some protection against possible exposure to cold temperatures after the product leaves manufacturing. Depending upon the exact formulation of the product, there may remain a slight possibility of precipitation of granules at temperatures very close to, but above 0° C., depending also on the length of exposure to cold and the degree of precipitation considered unacceptable.

The bone putty compositions of the invention may be transported using any variety of transportation methods widely known, including but not limited to commercial air cargo transportation. Transport in the cargo holds of aircraft can involve exposure, for certain periods of time, to relatively cold temperatures due to the properties of the upper atmosphere, or to other temperatures which may not be well controlled. It is also possible for other forms of shipment, such as ground transport, to involve exposure, for certain periods of time, to temperatures which are cold or not well controlled. Accordingly, the packaging for the bone putty composition of the present invention may include thermal protective materials.

The packaging in which the bone putty compositions of the invention are packaged is designed to maintain packaged bone putty composition above a critical temperature for a period of time, if the packaged bone putty composition has an initial temperature of about room temperature but is subsequently exposed to a cold external temperature. The packaging, in other words, may slow or delay a thermal transient experienced by the bone putty composition on exposure to external cold conditions.

The packaging may include thermal protective packaging which may include materials which offer any one or more of thermal insulating properties, heat capacity properties or phase change thermal properties. Packaging materials known in the art as having thermal insulating properties include, but are not limited to, foam, fibrous material, and bubble wrap. Packaging materials having heat capacity properties may include almost any solid or liquid material of suitable mass so as to provide a suitable heat capacity in terms of sensible heat. Packaging materials having phase change properties may include, but are not limited to, liquids or aqueous gels known in the art as having latent heat of phase change properties at a particular temperature or temperature range. Aqueous gel packaging includes, for example, Koolit Gel Packs (available from Cold Chain Technologies, Holliston, Mass.), which have a substantial thermal mass and undergo a phase change at a designated temperature such as about 0° C.

Any individual or combination of types of packaging having similar physical properties may be used with the bone putty compositions of the invention. Any particular form or substance of thermal protective packaging may have more than one of these properties. Layers of packaging having different properties may be used. Any such thermal protective packaging may be configured so as to substantially surround or nearly surround the packaged bone putty composition.

In applications other than the present application, the intended use of phase change products has often been as a source of cold temperatures to keep packaged articles chilled against exposure to warm temperatures. In the present invention, however, phase change products such as an unchilled gel pack may be used by being placed in packaging when they are at about room temperature (i.e., in an unfrozen condition), with the intention that the bone putty composition is insulated from the effects of cold temperature by the phase change products. If the thermal protective packaging is exposed to sub-freezing temperatures, the phase change material helps to delay the exposure of the article to cold temperatures first by the thermal mass of the material and then by the phase change. If the thermal protective packaging is exposed to cold temperatures which are above freezing, the phase change material helps to delay the exposure of the article to cold temperatures simply by virtue of its thermal mass.

It is believed that the use of a gelatinous material in the thermal protective packaging, rather than an ordinary liquid, may help to suppress convective motion of fluid within the packaging during a thermal transient, which may further help to slow the inward progression of such temperature changes toward the packaged article. With thermal protective packaging as described here, assuming the putty may have a small temperature region at cold temperatures in which the putty is vulnerable to precipitation of granules, it is possible for the package as a whole to be exposed to those temperatures or even colder temperatures for modest periods of time without the putty product itself experiencing those temperatures. Hence, the putty would not experience precipitation of its granules.

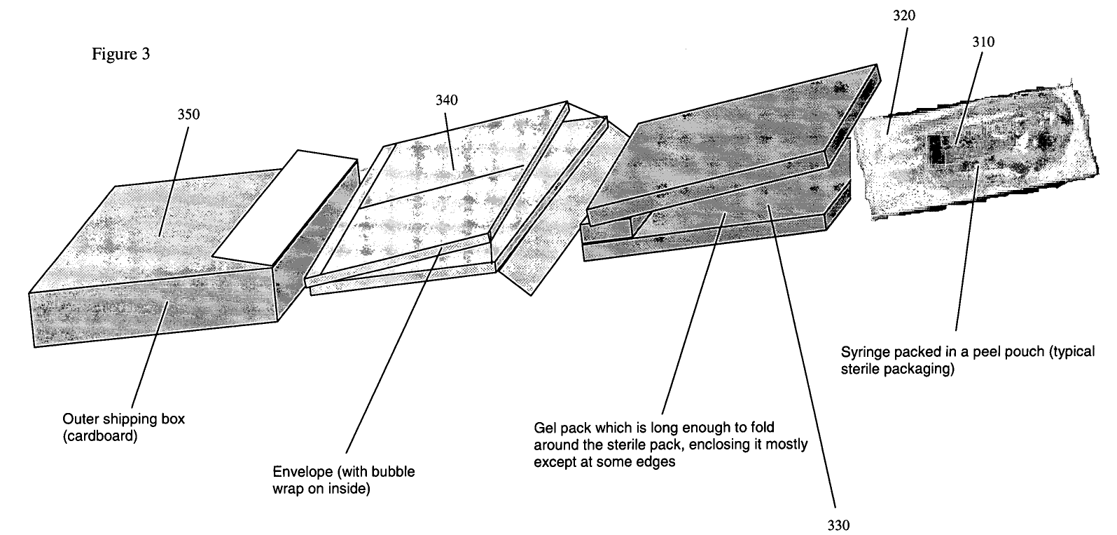

One embodiment of thermal protective packaging useful with the invention is illustrated in FIG. 3. The innermost article presented in FIG. 3 may be the bone putty composition itself, which may be packaged in a syringe, jar or other suitable container 310. The container 310 of the bone putty composition may then be enclosed within a sterile barrier package 320, such as a peel pouch. The sterile barrier package 320 may then be partially or completely surrounded by a thermal protection packaging 330 such as the gel pack described herein. The thermal protection packaging 330, together with the container 310 bone putty composition and its associated immediate packaging 320, may then be placed inside an envelope 340 which may include bubble wrap or other forms of mechanical protection or thermal insulation. The envelope, together with the enclosed packaging and compositions, may then be enclosed inside an external shipping container 350, such as a plastic or cardboard box.

In another embodiment, the invention provides a method of shipping or transporting a granule suspension at temperatures below about ambient or about room temperature, wherein the suspended granules do not precipitate from the carrier. The shipping or transporting method comprises formulating granules in a bone putty composition comprising a carrier formulation comprising a carrier substance present at a concentration of between about 35% to about 40% by weight of the carrier formulation, the balance of the bone putty composition being granules and any additional therapeutic agents.

In one embodiment, the bone putty composition comprises the carrier substance present at a concentration of about 35% by weight of the carrier formulation, if it is believed that the bone putty composition will not experience temperatures below about 10° C. for any substantial duration of time. In another embodiment, the bone putty composition comprises the carrier substance present at a concentration of about 37.5% by weight of the carrier formulation, if it is believed that the bone putty composition will not experience temperatures below about 5° C. for any substantial duration of time. In a further embodiment of the invention, the bone putty composition comprises the carrier substance present at a concentration of about 40% by weight of the carrier formulation, if it is believed that the bone putty composition will not experience temperatures below about 0° C. for any substantial duration of time.

The carrier substance may be any substance capable of maintaining substantially consistent gel-like rheological properties between about body temperature and about ambient or about room temperature, as well as to temperatures above about 0° C. The carrier substance may be a block copolymer, such as for example a poly(oxyalkylene) block copolymer. In one embodiment of the invention, the poly(oxyalkylene) block copolymer may be a poly(oxyethylene)-poly(oxypropylene)-poly(oxyethylene) triblock copolymer. In one embodiment of the invention, the carrier substance is Poloxamer 407 (Pluronic F127). In another embodiment of the invention, the carrier substance is selected from any one of the following, or combinations thereof: Poloxamer 188 (Pluronic F68); Poloxamer 237; Poloxamer 338; Poloxamer CRL-1072; Pluronic L61; Pluronic P85; Pluronic F88; Pluronic F98; Pluronic F108. Other variations of the triblock copolymer molecule are also possible, as are any combination of such substances.

The bone putty composition of the invention may further be useful for the treatment of various diseases and/or disorders of the human body. The bone putty composition of the invention may be useful to fill voids or defects in endogenous bone resulting from various causative agents, such as for example bone fractures. Examples of circumstances in which the bone putty compositions of the invention may be useful include, but are not limited to, the repair of defects or compromised bones resulting from fractures, surgery (including sites of implantation of bone screws), and infection. In these instances, the bone putty composition may be useful as a bone void filler that is applied to the affected bone, while also promoting the ingrowth of new bone tissue and other tissues such as vascular tissue.

The circumstances in which the bone putty composition of the invention may be used for treating diseases or disorders include, but are not limited to, aiding in the treatment of the adverse effects of genetic defects such as osteogenesis imperfecta. The bone putty composition of the invention further may be useful for the treatment of dental cavities, particularly non-superficial dental cavities having limited exposure to oral fluids. Likewise, the bone putty composition of the invention may be useful as a void filler for treating cracked teeth, or at periodontal sites, or as a filler composition for filling of a socket following tooth extraction(s).

Bone fractures that may be treated with the compositions of the invention include, but are not limited to, bone fractures of bone selected from bones of ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus tarsal and metatarsal bones, and condyle origin, as well as one or more combinations thereof. The bone putty composition of the invention can further be used to treat cracks or to fill voids which may be surgically created, or may exist for any other reason.

The bone putty composition of the invention may further be combined with one or more additional compositions prior to administration to a patient. It will be recognized by one of skill in the art that the rheological properties of the compositions described herein may vary with the addition of other compounds, and likely will. However, in a non-limiting example of use of the bone putty composition of the invention, the bone putty composition is first transported to the site of use as an unmodified composition, and after delivery to the location in which implantation is to occur the bone putty composition is combined with one or more agents designed to assist in the delivery of the bone putty composition to a site of implantation via a syringe or any other appropriate surgical means. Non-limiting examples of agents designed to assist in the delivery of the bone putty composition include blood, bone marrow or platelet rich plasma, the addition of which is known in surgical practice. Use of the bone putty composition of the present invention may include providing adjacent fixation of bones.

### EXAMPLES

### Example 1

A batch of bone putty composition was made as described herein. Granules of TCP were prepared by granulating, in a fluidized bed granulator, particles of hydroxyapatite and calcium pyrophosphate precursor having an average particle size of about 5 micrometers (Cosmocel, Monterrey, Nuevo León, México) together with particles of lactose having an average particle size of about 60 micrometers (Pharmatose, DMV International, Veghel, The Netherlands). The mixture of the two substances was about 40% by weight lactose, 60% by weight hydroxyapatite and calcium pyrophosphate.

The granulation was performed in a fluidized bed granulator (Glatt Air Techniques, Ramsey, N.J.) in which a binder liquid consisting of an aqueous solution of polyacrylic acid was sprayed while the powder particles were blown and suspended in air that flowed in a vertically upward direction. The process was carried out for a suitable length of time to yield desirably sized agglomerates.

Subsequently, the agglomerates were sintered in a kiln under atmospheric air conditions. The sintering schedule included a gradual heat-up period, a peak temperature of approximately 1200° C. for 2 hours, and a cooldown period. During the heat-up period, the lactose porogen and the binder were burned out of the agglomerates. At temperatures near and at the peak temperature, the particles of hydroxyapatite and calcium pyrophosphate reacted to form a high percentage of beta tricalcium phosphate, and the individual powder particles also sintered together forming the final granules. The composition of the resulting tricalcium phosphate was greater than about 75% beta tricalcium phosphate. Because of the geometry in which the granules were spread during sintering, there was some (unintended) light sintering of some granules to other granules, in addition to the desired sintering of particles to other particles within each granule. These lightly sintered bonds between granules were later broken during the subsequent sieving operation (which included vibration of the sieves).

The resulting granules were porous with an average pore size of about 60 micrometers, with pores ranging from about 5 micrometers to about 800 micrometers, and a fractional porosity of about 60%. Many, although not all, of the resulting granules were in the desired size range (overall external dimension of the granule). The sintered granules were then size-classified by sieving through screens, and granules in the size range of about 106 micrometers to about 425 micrometers were kept for use in the bone putty composition.

Poloxamer 407 was used as the carrier substance. Dissolution of the Poloxamer 407 in water was performed using a double planetary mixer manufactured by the Charles Ross and Son Company, Hauppauge, N.Y. The composition of the carrier formulation was 37.5% by weight Poloxamer 407 (also known as Pluronic F127) (BASF, Mt. Olive, N.J.), the balance as water. Dissolution was accomplished by addition of enough Poloxamer 407 to attain a concentration of about 20%, followed by mixing, followed by addition of enough Poloxamer 407 to attain a concentration of about 30%, followed by mixing, followed by addition of the remaining amount of Poloxamer 407 and mixing for a longer period of time. All dissolution was carried out at temperatures in the range of about 1° C. to about 4° C. at a sub-atmospheric pressure (above the vapor pressure of the water). This minimized the presence of air bubbles in the carrier.

To produce the bone putty composition, the granules of TCP were added and a similar mixing operation was performed, also at cold temperatures of about 1° C. to about 4° C. and reduced pressure, to mix the granules throughout the carrier formulation. The TCP granules amounted to about 30.6% of the total weight of the bone putty composition. Then, the bone putty composition was discharged from the mixing container, at cold conditions (about 1° C. to about 4° C.) into cartridges, each of which was suitable for filling with a precision syringe filler (EZ-Mix FR, Dispensing Technologies, S. Lancaster, Mass.). The precision syringe filler, which was microprocessor controlled, was capable of dispensing volumes to an accuracy of +/−0.1 cm³. Filling of individual syringes by the syringe filler was performed at room temperature.

The bone putty composition was loaded into syringes having a volumetric capacity of either about 3 cm³ or about 5 cm³. The syringe included a luer lock fitting whose minimum open area was a circular orifice having a diameter of about 4 mm (0.157 inch). The bone putty composition in the syringes was sterilized by exposure to a minimum of about 19 kiloGray of gamma irradiation.

During use, a syringe tip having an inside diameter of about 3 mm (0.118 inch) and a length of about 32.4 mm (1.275 inch) may be attached to the luer-lock fitting. It was found that the force needed to expel the irradiated bone putty composition from this assembly, at a flowrate suitable to expel the contents within 10 to 15 seconds, at room temperature, was less than approximately 44 N (10 lbf). This was considered a reasonable amount of force.

Possible precipitation of granules from suspension in the bone putty composition was investigated by testing syringes containing the bone putty composition using a water bath maintained at controlled temperatures. Precipitation of granules from the bone putty composition did not occur upon extended exposure to any temperature above about 3.5° C. However, it was found that in a very narrow temperature range there was the possibility of precipitation. For example, at a temperature of about 2° C., exposure durations exceeding two hours resulted in some precipitation. Exposure to a temperature of about 0° C. resulted in precipitation after about a half hour. These results established guidelines for thermal protection by the packaging.

Syringes containing the bone putty composition were packaged in packaging comprising a sterile peel pouch that was substantially surrounded by a 616SB-2 Gel pack from Cold Chain Technologies (Holliston, Mass.). This Gel pack design, referred to as a saddle-bag, can fold in half as illustrated in FIG. 3 so as to substantially surround the contents on two sides, leaving only a minimal amount of edge space which is not actually surrounded by the gel pack. The gel pack was then enclosed in an envelope incorporating a slight amount of bubble-wrap, which in turn was surrounded by a cardboard box packaging.

Temperature transient measurements were taken by exposing the packaging to controlled conditions of refrigeration at known temperatures. Temperatures at the location of the product were monitored by a temperature data logger. It was found that the packaging as described had a thermal time constant of about two (2) hours when the temperature to which the package was exposed was above 0° C. (i.e., no phase change in the Gel-Pack).

It was also found that exposure of the packaging to sub-freezing temperatures resulted in a temperature transient at the location of the product exhibiting a hold or plateau period, presumably associated with phase change of the aqueous gel. Packages that were packed as described in this example were shipped round-trip by commercial overnight air transport from the East Coast to the West Coast of the United States during summer weather. Some shipments were on non-stop flights, while other shipments experienced intermediate take-offs and landings. The temperature transient inside the packaging at the location of the putty product was recorded by a data logger. It was found that the temperatures experienced inside this packaging, both for non-stop flights as well as flights having lay-overs, remained within ranges which were acceptable for avoiding precipitation of granules from the putty.

### Example 2

In this example, bone putty composition were produced with a carrier formulation containing about 40% Pluronic 407 by weight of the carrier formulation, the balance of the carrier formulation as water. The granules were mixed in to produce the bone putty composition as described elsewhere herein. This bone putty composition was resistant to precipitation of granules at a wide range of shipping temperatures, because the bone putty composition remained a gel to the temperature of about 0° C. or below. As this composition was chilled from room temperature to colder and colder temperatures, it had essentially no viscous-liquid region before freezing to a solid, or at least no region in which its viscosity permitted granules to precipitate. This composition froze to form a solid somewhere between about −5° C. and about −10° C. Accordingly, with this bone putty composition, the use of thermal protective packaging may be less important than for the composition of Example 1.

At the temperature at which the bone putty composition would be used, i.e., about room temperature or about body temperature, this bone putty composition was somewhat more viscous than the bone putty composition of Example 1, which used about 37.5% carrier substance in the carrier formulation. Accordingly, the bone putty composition of this example may be packaged in jars or in syringes which offer relatively less resistance to the discharge of the bone putty composition than syringes already described herein. However, some syringe designs will not be suitable for this bone putty composition due to the viscosity of this bone putty composition.

### Example 3

In this example, bone putty composition is formulated with a carrier substance present at a concentration of greater than about 35% Pluronic 407 by weight of the carrier formulation, and a suitable amount of a conventional substance, with the balance of the carrier formulation being water. A conventional substance is in general considered to mean a substance which becomes stiffer or more viscous as temperature decreases. A conventional substance may be a gel-forming substance such as hydroxypropylmethylcellulose (HPMC) which becomes stiffer or more viscous with decreasing temperature. Such a substance may be a liquid which has substantial viscosity by itself, which viscosity increases monotonically with decreasing temperature. Such a substance may be a water-soluble compound whose aqueous solution has a viscosity which increases monotonically with decreasing temperature. (A monotonic function is a function which is either entirely non-increasing or non-decreasing, having a first derivative which does not change sign). Even if the conventional substance is a gel-former (able to create a gel when in aqueous solution at appropriate concentration), the concentration of the conventional substance which is actually used may not be sufficient to form a gel by itself, but simply might be such that, in combination with the already-described reverse phase material(s), will provide a desired increase in the viscosity of the composition at temperatures near about 0° C.

Conventional substances such as HPMC increase the viscosity of a solution in which they are present, and specifically they would most strongly increase the viscosity of such a solution at cold temperatures. Accordingly, conventional gel forming substances may help prevent precipitation of granules at cold temperatures, while not making the bone putty composition appreciably more difficult to dispense at about room temperature or about body temperature. The amount of conventional gel forming substance, such as HPMC, may be chosen to provide a desired viscosity or gel characteristic of the putty at cold temperatures.

Other suitable conventional gel forming materials which may be used in the bone putty compositions of the invention include, but are not limited to, carboxymethylcellulose.

Still other suitable conventional gel-forming materials which may be used include alginate; pectin; collagen; gelatin; fibrin (fibrinogen and thrombin); fat; platelet rich plasma gel; hyaluronic acid; chondroitan sulphate; chitosan; methylpyrrolidinone chitosan; glycerol; glycerol esters of fatty acids such as glycerol monooleate, glycerol monopalmitostearate and glycerol monolinoleate; dextran; PEG-PLGA diblock or triblock copolymers; PEG-PLA diblock or triblock copolymers; poly(ortho ester); PEO-PPO-PAA graft-co-polymer; PVA-g-PLGA graft-polymers; polyphosphazenes; polyvinyl alcohol; PEGT-PBT co-polymers; polyacrylamide; hydrolyzed polyacrylamide; polyacrylic acid salt; poly(N-isopropyl acrylamide); agarose; polyvinylpyrrolidone; starch; hydroxyethylated starch; and hydrolyzed polyacrylonitrile. An example of a suitable conventional substance which is itself a liquid and is not itself a gel-former, is glycerol.

The granules may subsequently be mixed with the carrier formulation to produce the bone putty composition of the invention using techniques described elsewhere herein.

The invention, as described herein, provides a bone putty composition whose rheological properties are gel-like over a wide range of temperatures. For a particular carrier substance (Poloxmer 407) the boundary between gel rheology and liquid rheology is shown in FIG. 1 as a function of concentration and temperature. In contrast to other Poloxamer 407 bone putty formulations which have entered the liquid region at or slightly below room temperature, the carrier formulation of the present invention maintains gel-like properties through a wide range of temperatures that could be encountered in shipping, and for one formulation even maintains gel-like properties even down to a temperature at which it freezes solid. All of this helps to maintain suspended granules reliably in suspension even in the face of unknown temperature fluctuations that may be experienced during shipping and handling.

The invention may be practiced in ways other than those particularly described in the foregoing description and examples. Numerous modifications and variations of the invention are possible in light of the above teachings and, therefore, are within the scope of the appended claims.

The entire disclosure of each document cited (including patents, patent applications, journal articles, abstracts, manuals, books, or other disclosures) in the Background of the Invention, Detailed Description, and Examples is herein incorporated by reference in their entireties.

While the invention has been described with reference to particularly preferred examples and embodiments, those skilled in the art will appreciate that various modifications may be made to the invention without departing from the spirit and scope thereof.

What is claimed is:

1. A bone putty composition comprising granules suspended in a carrier formulation comprising water and a carrier substance, wherein the carrier formulation exhibits reverse phase rheological behavior in at least a portion of a temperature range between about body temperature and about 10° C. and wherein the bone putty composition exhibits gel-like rheological properties throughout the temperature range between about body temperature and about 10° C.

2. A bone putty composition comprising granules suspended in a carrier formulation comprising water and a carrier substance, wherein the carrier formulation exhibits reverse phase Theological behavior in at least a portion of a temperature range between about body temperature and about 5° C. and wherein the bone putty composition exhibits gel-like rheological properties throughout the temperature range between about body temperature and about 5° C.

3. A bone putty composition comprising granules suspended in a carrier formulation comprising water and a carrier substance, wherein the carrier formulation exhibits reverse phase rheological behavior in at least a portion of a temperature range between about body temperature and about 0° C. and wherein the bone putty composition exhibits gel-like rheological properties throughout the temperature range between about body temperature and about 0° C.

4. The bone putty composition of any one of claims 1, 2 or 3, wherein the carrier formulation has a room temperature viscosity or stiffness and a body temperature viscosity or stiffness, and the body temperature viscosity or stiffness is at least as large as the room temperature viscosity or stiffness.

5. The bone putty composition of any one of claims 1, 2 or 3, wherein the carrier formulation comprises a triblock copolymer.

6. The bone putty composition of claim 5, wherein the triblock copolymer is a poly(oxyethylene)-poly(oxypropylene)-poly(oxyethylene) copolymer.

7. The bone putty composition of any one of claims 1, 2 or 3, wherein the carrier substance consists of Poloxamer 407 present at a concentration of between about 35% to about 40% by weight of the carrier formulation.

8. The bone putty composition of any one of claims 1, 2 or 3, wherein the carrier substance comprises Poloxamer 407 present at a concentration of between about 35% to about 40% by weight of the carrier formulation.

9. The bone putty composition of claim 1, wherein the bone putty composition comprises Poloxamer 407 present at a concentration of about 35% by weight of the carrier formulation.

10. The bone putty composition of claim 2, wherein the bone putty composition comprises Poloxamer 407 present at a concentration of about 37.5% by weight of the carrier formulation.

11. The bone putty composition of claim 3, wherein the bone putty composition comprises Poloxamer 407 present at a concentration of about 40% by weight of the carrier formulation.

12. The bone putty composition of claim 1, wherein the granules remain substantially suspended in the bone putty composition throughout a temperature range between about 10° C. and about body temperature.

13. The bone putty composition of claim 2, wherein the granules remain substantially suspended in the bone putty composition throughout a temperature range between about 5° C. and about body temperature.

14. The bone putty composition of claim 3, wherein the granules remain substantially suspended in the bone putty composition throughout a temperature range between about 0° C. and about body temperature.

15. The bone putty composition of claim 1, wherein the carrier formulation further comprises a substance whose aqueous solution has a viscosity which monotonically increases with decreasing temperature throughout the temperature range from about body temperature to about 10° C.

16. The bone putty composition of claim 2, wherein the carrier formulation further comprises a substance whose aqueous solution has a viscosity which monotonically increases with decreasing temperature throughout the temperature range from about body temperature to about 5° C.

17. The bone putty composition of claim 3, wherein the carrier formulation further comprises a substance whose aqueous solution has a viscosity which monotonically increases with decreasing temperature throughout the temperature range from about body temperature to about 0° C.

18. The bone putty composition of claim 1, wherein the carrier formulation further comprises a substance which has a viscosity which monotonically increases with decreasing temperature throughout the temperature range from about body temperature to about 10° C.

19. The bone putty composition of claim 2, wherein the carrier formulation further comprises a substance which has a viscosity which monotonically increases with decreasing temperature throughout the temperature range from about body temperature to about 5° C.

20. The bone putty composition of claim 3, wherein the carrier formulation further comprises a substance which has a viscosity which monotonically increases with decreasing temperature throughout the temperature range from about body temperature to about 0° C.

21. The bone putty composition of claim 1, wherein the carrier formulation further comprises a gel-forming substance which in aqueous solution is capable of forming a gel whose stiffness monotically increases with decreasing temperature throughout the temperature range from about body temperature to about 10° C.

22. The bone putty composition of claim 2, wherein the carrier formulation further comprises a gel-forming substance which in aqueous solution is capable of forming a gel whose stiffness monotically increases with decreasing temperature throughout the temperature range from about body temperature to about 5° C.

23. The bone putty composition of claim 3, wherein the carrier formulation further comprises a gel-forming substance which in aqueous solution is capable of forming a gel whose stiffness monotically increases with decreasing temperature throughout the temperature range from about body temperature to about 0° C.

24. The bone putty composition of any one of claims 1, 2 or 3, further comprising a therapeutic agent.

25. The bone putty composition of claim 24, wherein the therapeutic agent is a BMP, a TGF-beta, a VEGF, an osteoclastogenesis inhibitor, or any combination thereof.

26. The bone putty composition of claim 25, wherein the therapeutic agent is a polynucleotide.

27. The bone putty composition of claim 25, wherein the therapeutic agent is a polypeptide.

28. The bone putty composition of any one of claims 1, 2 or 3, wherein the granules consist of tricalcium phosphate.

29. The bone putty composition of any one of claims 1, 2 or 3, wherein the granules comprise tricalcium phosphate.

30. The bone putty composition of any one of claims 1, 2 or 3, wherein the granules have a composition which is greater than about 50% by weight of beta tricalcium phosphate.

31. A method of shipping or transporting any one of the bone putty compositions of claims 9, 10 or 11, the method comprising formulating one or more of said bone putty compositions; packaging the one or more of said bone putty compositions in one or a plurality of containers; and shipping or transporting the one or more packaged bone putty compositions.

32. The method of claim 31, wherein the bone putty composition comprises granules which comprise tricalcium phosphate.

33. The method of claim 31, wherein the bone putty composition comprises granules which consist of tricalcium phosphate.

34. The method of claim 31, wherein shipping or transporting the bone putty composition comprises packaging the bone putty composition together with a thermal protective packaging comprising at least one of a thermal insulating substance, a thermal mass substance, and a phase change substance.

35. The method of claim 31, wherein shipping or transporting the bone putty composition comprises packaging the bone putty composition together with a thermal protective packaging which comprises a substance which is a gel during at least some temperatures to which the thermal protective packaging may be exposed during the shipping or transporting.

36. The method of claim 31, wherein shipping or transporting the bone putty composition comprises packaging the bone putty composition together with a thermal protective packaging which surrounds the bone putty composition on at least two sides.

37. A kit comprising at least one container comprising the bone putty composition of any one of claims 1, 2 or 3, and a syringe.

38. A method of making the bone putty composition of any one of claims 9, 10 or 11, comprising mixing granules of interest and a carrier formulation comprising water and the Poloxamer 407 as the carrier substance, wherein the mixing comprises adding the Poloxamer 407 in incremental amounts to the carrier formulation, and dissolving the added Poloxamer 407 into the carrier formulation between incremental additions, suitably to achieve the stated concentration of the Poloxamer 407.

39. The method of claim 38, further comprising terminally sterilizing the bone putty composition.

40. The method of claim 39, wherein the sterilizing comprises exposing the bone putty composition to radiation selected from the group consisting of electron beam irradiation, Gamma irradiation, and a combination thereof.

41. The method of claim 38, wherein the carrier substance contained in the bone putty composition has a molecular weight of about 13,000 to about 14,000 Daltons following sterilization.

42. The method of claim 38, wherein the carrier substance contained in the bone putty composition has a molecular weight of about 16,000 to about 19,000 Daltons following sterilization.

43. A method of treating a bone defect or bone fracture, comprising administering to a patient with a bone defect or bone fracture the bone putty composition of any one of claims 1, 2 or 3.

44. The method of claim 43, wherein the bone fracture is a fracture selected from ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus tarsal and metatarsal, and condyle bones, as well as one or more combinations thereof.

45. The method of claim 43, wherein the bone defect is a dental cavity or a periodontal region or a tooth extraction site.

46. The method of claim 43, wherein the carrier substance comprises Poloxamer 407.

47. The method of claim 43, wherein the carrier substance consists of Poloxamer 407.

48. A bone putty composition comprising granules suspended in a carrier formulation comprising water and a carrier substance, wherein the carrier substance comprises a triblock copolymer and wherein the bone putty composition exhibits gel-like rheological properties throughout a temperature range between about body temperature and about 10° C.

49. A bone putty composition comprising granules suspended in a carrier formulation comprising water and a carrier substance, wherein the carrier substance comprises a triblock copolymer and wherein the bone putty composition exhibits gel-like Theological properties throughout a temperature range between about body temperature and about 5° C.

50. A bone putty composition comprising granules suspended in a carrier formulation comprising water and a carrier substance, wherein the carrier substance comprises a triblock copolymer and wherein the bone putty composition exhibits gel-like rheological properties throughout a temperature range between about body temperature and about 0° C.

51. The bone putty composition of any one of claims 43, 44 or 45, wherein the carrier formulation comprises a triblock copolymer which comprises a poly(oxyethylene)-poly(oxypropylene)-poly(oxyethylene) copolymer.

52. A method of making a bone putty composition comprising granulating particles of a decomposable porogen and particles of precursors of tricalcium phosphate together with a decomposable binder substance to form agglomerates; heating the agglomerates to a temperature suitable to decompose the decomposable porogen and the decomposable binder; heating the agglomerates to a temperature suitable to cause the precursors to react to form tricalcium phosphate; heating the agglomerates to a temperature suitable to cause the agglomerates to sinter to form porous granules; and suspending the porous granules in a carrier formulation.

53. A method of making a bone putty composition, comprising granulating particles of a decomposable porogen and particles of a ceramic material together with a decomposable binder substance to form agglomerates; heating the agglomerates to a temperature suitable to decompose the decomposable porogen and the decomposable binder; heating the agglomerates to a temperature suitable to cause the agglomerates to sinter to form porous granules; and suspending the porous granules in a carrier formulation.

54. A method of making a bone putty composition, comprising granulating particles of precursors of tricalcium phosphate together with a decomposable binder substance to form agglomerates; heating the agglomerates to a temperature suitable to decompose the decomposable binder; heating the agglomerates to a temperature suitable to cause the precursors to react to form tricalcium phosphate; heating the agglomerates to a temperature suitable to cause the agglomerates to sinter to form porous granules; and suspending the porous granules in a carrier formulation.
